(19) United States
(12) Patent Application Publication
(10) Pub. No.: US 2019/0192065 A1
(43) Pub. Date: Jun. 27, 2019

(54) Systems for Testing and Enhancing Skin Health

(71) Applicant: Le CultureClub Inc., New York, NY (US)

(72) Inventor: Mandi Nyambi, Brooklyn, NY (US)

(21) Appl. No.: 16/231,839

(22) Filed: Dec. 24, 2018

Related U.S. Application Data
(60) Provisional application No. 62/610,133, filed on Dec. 22, 2017.

(51) Int. Cl. A61B 5/00; G16H 50/20; C12Q 1/689; A61K 35/741; C12Q 1/686

(52) U.S. Cl. CPC A61B 5/441; G16H 50/20; C12Q 1/6883; A61K 35/741; C12Q 1/686; C12Q 1/689

(57) ABSTRACT

New systems, devices and methods for testing, analyzing and managing skin and skin microbiomes are provided. According to some embodiments, a skin and skin microbiome testing and management system is provided, including at least one computer hardware- and software-based control system and a wand device with rapidly replaceable testing and application cartridges comprising rollers. In some embodiments, the control system enrolls user(s) and issues testing kits to the user(s), including the specialized wand device, testing substrates and reagents. In some embodiments, techniques for sampling and testing a skin microbiome are set forth. Further techniques for analyzing skin conditions, based on the results of that testing, and issuing management techniques based on those results, are also provided.

### CROSS-REFERENCE TO RELATED APPLICATIONS

This application claims the benefit of priority to U.S. Provisional Application No. 62/610,133, filed Dec. 22, 2017, titled “Systems for Testing and Enhancing Skin Health” which is incorporated herein by reference in its entirety.

### TECHNICAL FIELD

The present invention relates to dermatological testing and skin management and, more particularly, to devices, systems and methods for sampling, analyzing and managing human skin, and microbes present within human microbiomes.

### BACKGROUND

All mammals, including humans, have skin—a flexible exterior organ that covers, supports and protects the body from the exterior environment. Skin is the largest organ in the human body, and performs a number of important roles essential to overall health, ranging from providing a barrier against infection to regulating hydration. Human skin has several layers, including a lower or internal layer (the “dermis”) and an upper or exterior layer (the “epidermis”).

Human skin includes a flora of living microorganisms known as a skin microbiome, mostly held in the epidermis. The composition of the microbiome varies, depending on a number of factors. For example, ordinarily, over 1,000 species of bacteria are present on a person's skin, in very large numbers and varying proportions (greatly exceeding the number of human cells within the person's body). Although at least some human skin microbiomes certainly contain pathogenic bacteria, and bacteria has historically been equated with infection in everyday parlance, bacteria of the skin microbiome are often harmless, and often even beneficial. Human skin microbiomes may contain a significant number of beneficial species of bacteria or other microorganisms. Sometimes, at least arguably, these beneficial species may even form a part of the human organism, through mutualistic relationships that drive off infections or supply nutrients to the host person. In some instances, by contrast, bacteria are harmful, creating dangerous infections that damage human tissue. In any event, the particular composition of a skin microbiome may impact and reflect some health and aesthetic qualities of that person's skin, not to mention some aspects of the person's overall wellbeing.

The field of probiotics deals with introducing helpful bacteria into microbiomes, such as the human skin microbiome. Prebiotics, by contrast, deal with techniques that fertilize that probiotic bacteria, encouraging them and their beneficial effects to proliferate. Although results have varied, some probiotic and prebiotic techniques have shown great promise.

It should be understood that the disclosures in this application related to the background of the invention, such as the matter set forth in this section titled “Background,” are to aid readers in comprehending the invention, and do not simply set forth prior art or other publicly known aspects affecting the application; instead, the disclosures in this application related to the background of the invention comprise details of the inventors' own discoveries, observations, and other work and work results, including aspects of the present invention. Nothing in the disclosures related to the background of the invention is or should be construed as an admission related to prior art, or the work of others prior to the conception or reduction to practice of the present invention.

### SUMMARY

New systems, devices and methods for testing, analyzing and managing skin and skin microbiomes are provided. According to some embodiments, a skin and skin microbiome testing and management system is provided, including at least one computer hardware- and software-based control system and a sampling swab (e.g., a specialized wand device with rapidly replaceable testing and application cartridges comprising rollers). In some embodiments, the control system enrolls user(s) and issues testing kits to the user(s). In some embodiments, the kits include testing substrates, media and reagents. In some such embodiments, such kits may include such a specialized wand device. In other embodiments, such kits may include any commercially available swab suitable for use in sampling a human skin microbiome. In some embodiments, techniques for sampling and testing a skin microbiome are set forth. In other embodiments, the system may then receive samples and analytes obtained from the user(s), and then send skin management techniques to the user based on DNA testing of those samples and analytes, among other aspects. In some such embodiments, the system may send product or other recommendations to the user, which the user can then follow or purchase through the system. In some such embodiments, a wide array of commercially available products for skin care may be presented through the system. For example, in some such embodiments, commercially available products are presented to the user via an e-commerce module or other GUI aspect provided through a control system comprising a computer hardware and software. Further techniques for analyzing skin conditions, based on the results of that testing, and issuing management techniques based on those results, are also provided, in some embodiments.

Further aspects of the invention will be set forth in greater detail, below, with reference to the particular figures.

### BRIEF DESCRIPTION OF THE DRAWINGS

The features and advantages of the example embodiments of the invention presented herein will become more apparent from the detailed description set forth below when taken in conjunction with the following drawings.

FIG. 1 is a perspective drawing of an example wand device, which may be used to sample and manage a skin microbiome, according to some embodiments of the present invention.

FIG. 2 is a process flow diagram, setting forth several example steps that may be undertaken by a user and/or control system implementing some skin microbiome sampling and skin management embodiments of the present invention.

FIG. 3 is a schematic block diagram of some elements of an example control system, preferably incorporating a machine-readable medium, that may be used to implement various aspects, in accordance with some embodiments of the present invention.

FIG. 4 is a perspective drawing depicting a user and specialized hardware carrying out example aspects of a method of sampling and managing human skin and a microbiome, according to some embodiments of the present invention.

FIG. 5 depicts an example kit which may be issued to, and used by, a user of a skin testing and management system and method, according to some embodiments of the present invention.

FIG. 6 depicts a table, coding and other aspects of an example array of primers for DNA amplification and testing of a sample taken from a human skin microbiome, according to some embodiments of the present invention.

FIG. 7 is a process flow diagram, setting forth several example steps that may be undertaken by a user and/or control system implementing some skin microbiome sampling and skin management embodiments of the present invention.

### DETAILED DESCRIPTION

The example embodiments of the invention presented herein are directed to systems, devices and methods for testing, analyzing and managing skin and skin microbiomes. As mentioned above, in some embodiments, a skin and skin microbiome testing and management system, including at least one computer hardware- and software-based control system, enrolls user(s) and issues testing kits to the user(s). In some embodiments, such kits include a sampling device. In some such embodiments, a sampling device is a specialized wand device with rapidly replaceable testing and application cartridges comprising rollers. In other embodiments, such kits include any commercially available swab suitable for use in sampling a human skin microbiome. In some embodiments, swabs comprise a substrate suitable for holding sampling or application media. In some embodiments, such a sampling or application media comprises a liquid. In some embodiments, such a media is provided to a user loaded within the substrate. In other embodiments, such a media is provided to the user separately from the swab and substrate (e.g., in a separate vial). In some embodiments, such a media comprises nutrients or other ingredients beneficial for human skin. In other embodiments, such nutrients or other ingredients are supplied separately, or added to such media. In some embodiments, a testing kit issued to a user includes instructions for conducting the sampling of her or his skin microbiome. In some such embodiments, a user carries out such instructions, and sends samples and analytes of her or his skin microbiome (e.g., from particular region of her or his face) to the control system. In some embodiments, the system may then receive samples and analytes obtained from the user(s), and then send skin management techniques to the user based on DNA testing of those samples and analytes, among other aspects. In some such embodiments, the system may send product recommendations to the user, which the user can then purchase through the system. In some such embodiments, a wide array of commercially available products for skin care may be presented through the system. For example, in some such embodiments, commercially available products are presented to the user via an e-commerce module or other GUI aspect provided through a control system comprising a computer hardware and software. In some embodiments, the control system issues other recommendations to the user, relevant to her or his skin health. In some embodiments, the control system issues other recommendations to the user, relevant to her or his skin appearance. In some embodiments, the control system requests that a user select a goal relevant to her or his skin health or appearance. In some embodiments, after receiving and testing samples from such a user, the control system indicates to a user whether she or he is making progress toward a particular goal, based on results of that testing. In some such embodiments, if the user is making progress, according to signs of skin health correlated with such a goal that are the subject of that testing, the control system reports that progress to the user. In some such embodiments, if the user is not making progress, according to signs of skin health correlated with such a goal that are the subject of that testing, the control system reports that lack of progress to the user. In some embodiments, the control system may issue additional recommendations to the user, based on such progress or lack of progress. Additional techniques for analyzing skin conditions, based on the results of that testing, and issuing management techniques based on those results, are also provided.

This description is not intended to limit the application of the example embodiments presented herein. In fact, after reading the following description, it will be apparent to one skilled in the relevant art(s) how to implement the following example embodiments in alternative embodiments.

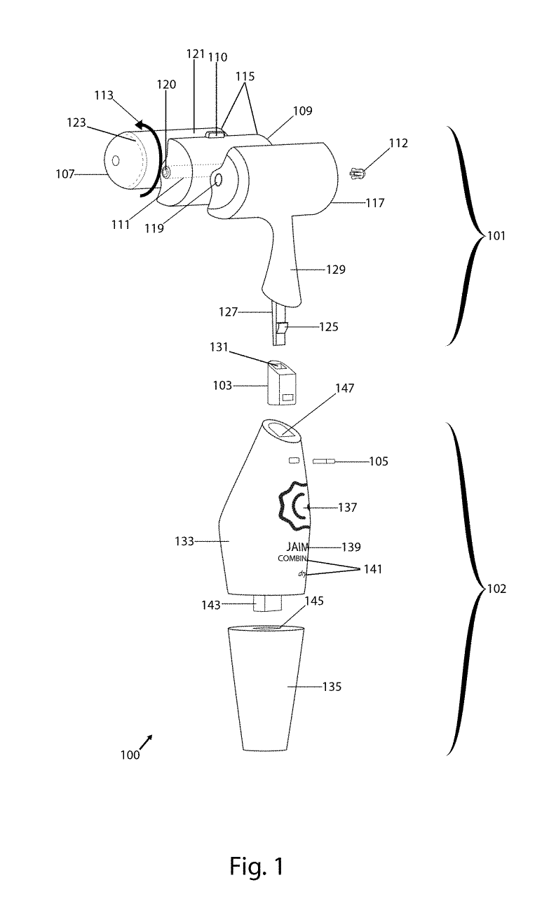

FIG. 1 is a perspective drawing of an exemplary device, which will be referred to as a wand device 100, which may be used to sample and manage a skin microbiome, in accordance with some embodiments of the present invention. Wand device 100 is illustrated in a partially exploded view, to illustrate certain exemplary components, each of which will be discussed in detail, below.

In some embodiments of the invention, wand device 100 may be comprised of at least two major sections: (1) a rolling applicator section 101 and (2) a handle grip section 102. In addition, wand 100 may comprise reversible connection hardware, such as an intermediate connection hub 103 and peg and/or release button 105, which aid in reversibly joining and decoupling rolling applicator section 101 and handle grip section 102, at any time chosen by a user, manufacturer or repair person.

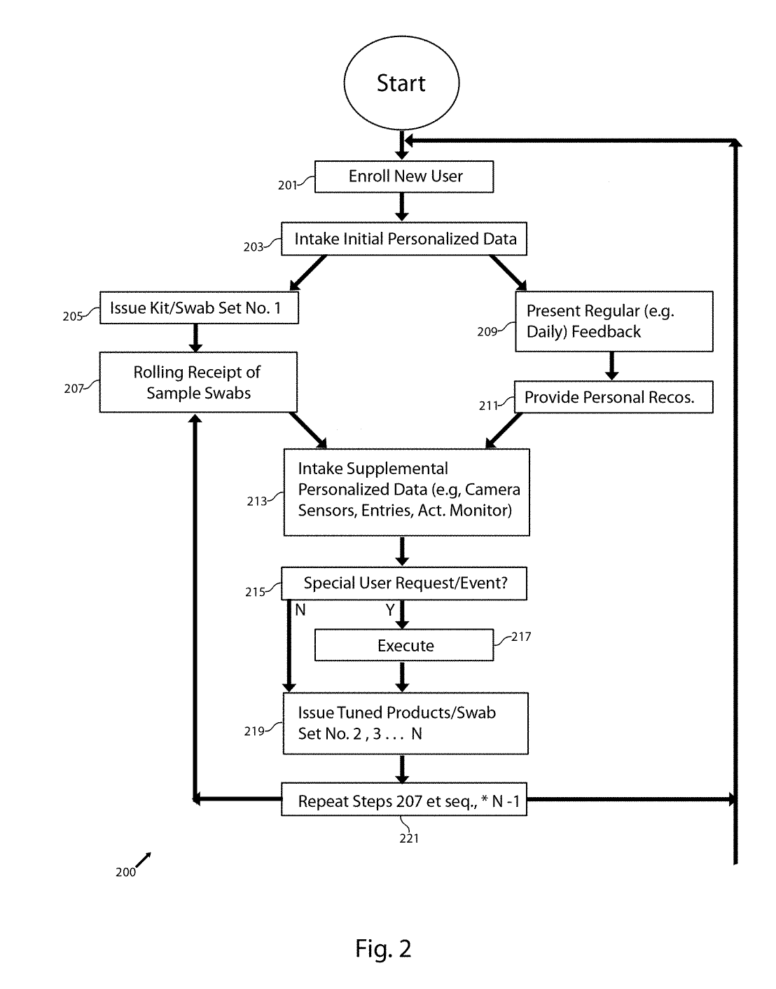

Rolling applicator section 101 may be configured for directly contacting, sampling materials from and managing skin (and, more specifically, a skin microbiome) with a rolling action, through methods set forth in greater detail below, in reference to FIG. 2. A sampling and/or application roller 107 may be variably, reversibly fastened or decoupled to a protective cartridge case 109, via a rotational axel 111 and locking end cap 112, about which sampling and/or application roller 107 may freely rotate, as shown by exemplary rotational motion arrow 113 (or, in some embodiments, in the opposite rotational direction). In some embodiments, such a sampling and/or application roller includes is rounded, as pictured. Protective cartridge case 109 provides a protective housing that at least partially covers the sampling and/or application roller 107, and itself may be variably, reversibly fastened or decoupled to a cartridge holder 117, using a grip-enhancing, projecting tab 110. Preferably, sampling and/or application roller 107 is delivered to a user pre-assembled, coupled with protective cartridge case 109 as a greater cartridge unit 115. The greater cartridge unit 115 then may be installed onto cartridge holder 117 of the wand device 100 preferably by two reversible axial snaps or dimpled fasteners, such as the example shown as 119, that grip complementarily shaped convex surface protrusions, such as the example shown as convex surface protrusions 120 of greater cartridge unit 115, such that the greater cartridge unit 115 can be changed out from cartridge holder 117, preferably creating an audible click or snapping sound upon installation, reassuring the user that the greater cartridge unit 115 has been properly installed onto cartridge holder 117. Thus, by providing a user with a set or other supply of similarly shaped and configured cartridge units, similar to example greater cartridge unit 115, a user of wand device 100 may install and uninstall several such cartridge units onto and off of cartridge holder 117 (and, thereby, wand device 100), and wand device 100 may therefore be used to take several samples of a user's skin microbiome with rollers, such as sampling and/or application roller 107, and deliver testing or application media, nutrients, chemicals, probiotics or other beneficial materials held on such rollers to the user's skin, in accordance with methods set forth in the present application.

Sampling and/or application roller 107 may be provided with a wide variety of rolling surface materials 121, which preferably create a rounded surface for sampling and/or application roller 107, as pictured. In some embodiments, rolling surface materials 121 may comprise a sampling or application substrate 123, impregnated with a testing or application media, which media preferably comprises a liquid. In any event, the rolling surface materials 121 may directly contact a user's skin, rolling over the surface of her or his face, until a substantial area of her or his face has been sampled or covered with an agent. To aid in sampling a user's microbiome, sampling or application substrate 123 preferably is covered with a plurality of small fibers or protrusions, greatly increasing the contact surface area to be rolled against, and swab, a user's skin—collecting a sample of the skin microbiome. The surface of sampling or application substrate 123 may be highly involuted and may comprise reticulated and/or capillary formations for gathering and holding samples captured through the methods set forth in this application. Similar substrate surface configurations may aid in holding a testing or application media for management of the user's skin, in which event they aid in holding larger amounts of the media, and automatically release it under the pressure of contact with the user's skin. In some embodiments, as will be discussed further below, sampling and/or application roller 107, and sampling swabs in general, may be provided to users dry, meaning that they may not, initially, be impregnated or include a testing or application media in or on their substrate or surface materials. In some such embodiments, a user may be instructed to douse or immerse such swabs, prior to use on his or her skin—for example, by providing the user with a bottle or vial of testing or application media and/or agents. Such uses may include sampling the user's microbiome, in some embodiments. Such uses may include applying an agent to the user's skin and microbiome, in some embodiments. Such uses may include sampling the user's microbiome and applying an agent to the user's skin and microbiome, in some embodiments.

Generally, the rolling design of rollers, such as sampling and/or application roller 107, also ensure an even distribution of a testing or application media, and prevent contamination of the media and/or substrate with areas other than the surface of the skin managed by the invention. In addition, sampling by rollers in accordance with aspects of the present invention is similarly protected from cross-contamination. The protective cartridge case 109 and handle grip section 102 similarly prevent inadvertent contamination of rollers such as sampling and/or application roller 107 (e.g., contamination caused by handling or dropping).

The particular type of substrate and testing or application media used in or with sampling and/or application roller 107 is preferably custom-made for the user's skin type or classification, and other data related to the user's skin, based on all data collected from the user, in accordance with aspects of the present invention. In cartridges designated for skin condition management, the testing or application media held in sampling or application substrate 123 may comprise nutrients, prebiotics, probiotics and other aspects or elements, according to a formulation determined to be suitable or ideal for the particular user, or type of user (e.g., based on the user's skin type or other skin and/or user classification), at the time of application. As mentioned above, this testing or application media may be provided to a user within the substrate in some embodiments. However, in some embodiments, the sampling or application substrate 123 may be provided to a user “dry,” meaning that it is provided separately from testing or application media, to a user (e.g., in a bottle or vial). In such cases, the sampling or application substrate 123, and the greater cartridge unit 115 in which it is provided, serves to spread the media and cover the user's skin, but may not contain the media initially, when issued to the user. The user's skin type may be determined by a variety of factors and data supplied by the user and data determined from testing, as discussed in greater detail below, and including the composition of microbes hosted in or on their skin. The testing or application media may comprise probiotic microbes, in a particular formulation and density personalized for a particular user and the particular user's skin type, and in an amount designated for a single application of the roller to a user's face. The composition, and/or composite ratio, of microbes (i.e., their relative presence within a media formulation within a cartridge, designated for the user) may be modulated to address particular needs of the user, during the testing steps set forth below (including, but not limited to, analyses of samples taken from cartridges sent to the user, at least in part, for testing). Prebiotics, nutrients, vitamins, and other growth-supporting and health-, hygiene and appearance-enhancing media ingredients and aspects (which may be naturally occurring or synthetic) may be included in the particular formulation of testing or application media supplied to the user in a cartridge designated for application to that user. For example, a format of skin management for a particular user may be a greater cartridge unit 115 or regimen of such cartridge units (or, in some embodiments, more conventional swabs or a regimen of such swabs) and particular applications thereof, incorporating an application media designated as a cleanser, moisturizer and/or serum for the user. If those greater cartridge units (and sampling and/or application rollers within them) or, in some embodiments, more conventional swabs, are sent to a user for testing, the substrates of those greater cartridge units or more conventional swabs preferably comprises flocked nylon swab materials, of a nature developed by COPAN Diagnostics, Inc. for conducting microbiology assays and other test sampling. Such substrates may incorporate capillary structures that may prevent the absorption of at least some microbes into the interior of those sampling and/or application rollers or swabs, while aiding in the elution of samples into a lysis buffer, from which genetic material (and specifically, genetic material of microbes hosted by a user's skin) may be extracted by technicians and scientists (and/or, in some embodiments, testing robots or other automated testing machinery) controlled by a user, administrator and/or control system carrying out steps in accordance with the present invention.

It should be understood that, throughout this application, steps of particular methods set forth may be conducted either by a human administrator, or a control system, and the ability of either type of agent to conduct such steps will be apparent to a person of ordinary skill in the art. Thus, the recitation of a user-, administrator-, or control system-conducted step also should be understood to include the execution of that step by any of the other possible types of agent, where possible.

In some embodiments, as mentioned above, swabs are provided in the rolling format of a generally cylindrical, rotating swab, attached to a handle grip section 102. Generally speaking, the handle grip section 102 allows the user (and particularly, a lay user) to apply the sampling and/or application roller 107 smoothly and evenly across her or his skin, without the contamination that would otherwise occur by directly handling a swab. In some embodiments, a plurality of different greater cartridge units designed for sampling and testing a human skin microbiome (“test cartridge(s)”) (with different sampling and/or application rollers, such as 107) different sampling or application substrates and different sample-gathering media (a.k.a. a “testing media”), or designated for different tests) may be provided to a user for testing his or her skin microbiome by swabbing. In one embodiment, a set of three different test cartridges is provided to a user (at least initially), as a set of cartridges for testing. (In some embodiments, wherever a test cartridge is stated in this application to be used to implement aspects of the invention, a more conventional swab for testing a human skin microbiome may instead be used.)

As mentioned above, handle grip section 102 is reversibly physically fastened together with rolling applicator section 101 with intermediate connection hub 103 and peg or release button 105 (preferably, connected to a latching switch configured to couple and decouple intermediate connection hub 103 from other parts of wand device 100) and force-biased with a spring. In some embodiments, a single fastener, an adhesive and/or built-in fasteners may alternatively, or additionally, be used to connect sections of wand device 100. In still other embodiments, all or some parts or sections set forth in this figure are, instead, permanently joined, or machined as a single, continuous part. The particular form and arrangement illustrated in the figure is exemplary only of the infinite possible variations of parts and their arrangements that are possible, within the scope of the invention, as will be readily apparent to those of skill in the art. As an example of a built-in connector, cartridge holder 117 may comprise a locking tab 125 on the surface of a tenon 127, at the base of an arm 129 of cartridge holder 117. Tenon 127 may then be threaded into a complementarily-shaped mortise notch 131, in the form of a notch-gripping hole, within another component of wand device 100 (such as intermediate connection hub 103, as pictured, or, in other embodiments, directly in a part of handle grip section 102). Handle grip section 102 may comprise two sub-components: a personalized neck 133, and a main handle 135. Preferably, personalized neck 133, or another component of wand device 100, is etched, labeled, or otherwise marked with both the branding of a company or other agent managing the invention, and with identifying information for the user and/or skin type/skin regiment type being sampled and/or applied with an agent by the wand device 100 (and, in some embodiments, by the methods set forth in the present application). For example, personalized neck 133 is pictured with a label, such as brand or logo 137, as well as the user's name 139 (Jaime Kyle, in this example) and skin type or classification 141 (“Combination Skin,” and “dry,” as pictured), which designations correspond with a formulation of testing or application media and/or type of substrate, roller and cartridge unit (a.k.a., “cartridge”), as discussed above, which is suitable for that skin type or classification. In some embodiments, however, wand device 100 may not include a label. In other embodiments, the label may be in a different form, and include different information than that pictured in the present figure. For example, in some embodiments, the user's name may not be printed on the label. In some embodiments, the user's skin type may not be printed on the label. In some embodiments, a different, unconventional system of skin type classification may be exhibited, incorporating a new, non-standard coding or nomenclature not previously known in the art.

Each of the parts set forth for the exemplary wand device 100, set forth above, and for other embodiments of devices set forth in the present application, may be manufactured by any methods known in the art, including, but not limited to, custom milling, injection molding, and automated assembly in mass production, incorporating any suitable material known in the art, including, but not limited to, plastics, metals, rubbers, textiles, adhesives, glasses, minerals and wood. For example, personalized neck 133 and main handle 135 may be joined by an adhesive, strengthening the joint between tenon 143 and mortise 145, when they are brought together in assembly. Similarly, peg and/or release button 105 may be omitted in some embodiments, with intermediate connection hub 103 glued or welded both to personalized neck 133 (at mortise 147) and/or to tenon 127, in some embodiments. Manufacturing of these parts, and the completed wand, in some instances, may incorporate off-the-shelf parts, while, in other instances, exclusively custom-made parts may be used. Preferably, at least part of wand device 100 comprises copper, another copper-like substance, injection-molded high-density polyethylene, and/or an acrylic polymer like Dupont's CORIAN® surface material, and/or other materials suitable for regular handling and use with little or no degradation. In some embodiments, some parts, such as intermediate connection hub 103, protective cartridge case 109, cartridge holder 117, and personalized neck 133, are constructed from injection-molded high-density polyethylene, while other parts are made from milled CORIAN and/or aluminum. For example, in some embodiments, peg and/or release button 105 is constructed from aluminum, and main handle 135 is constructed from milled CORIAN.

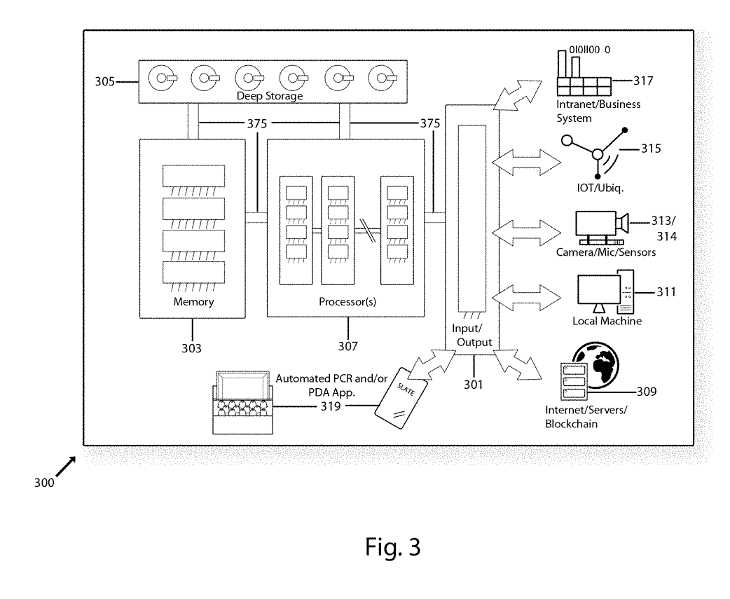

FIG. 2 is a process flow diagram, setting forth several exemplary steps 200 that may be undertaken by a user, administrator and/or a control system (such as the control system set forth below, in reference to FIG. 3) implementing some skin microbiome sampling and skin management embodiments of the present invention using a sampling and application device, such as the wand device 100 set forth above, in reference to FIG. 1.

In some embodiments according to the invention, an interval-covering supply of cartridges, such as greater cartridge unit 115, discussed above (or, in some embodiments, other swabs, as discussed in greater detail below), is sent to the user at regular intervals. For example, in a preferred method for distribution of the roller cartridges or other swabs, a set of the cartridges serving as a 30-day supply is sent to a user every 30 days, or a month-long supply is sent to a user each and every month, and the receiving user then simply applies one of those cartridges each day to his or her face, cleansing and/or managing her or his skin. In other embodiments, a weekly supply is provided to a user, e.g., once per week. In other embodiments, a quarterly supply is supplied to a user four times per year. In various other embodiments, any other amount of cartridges may be supplied at any suitable intervals, which may vary. In other embodiments, a supply of other swabs, covering similar intervals, is supplied to the user.

As mentioned above, in some aspects of the invention, a control system monitors results of aspects of the invention and delivers feedback to a user. Preferably, that feedback, along with other information, is provided in a web-based application, in some embodiments. In some embodiments, such an application may be a mobile application (e.g., on provided on a user's smartphone). In some embodiments, the application may remind a user to apply the wand device 100 and particular cartridges. In other embodiments, the application may remind a user to use particular skin care products. In some such embodiments, such skincare products may be, and may have been, purchased through the application. In some embodiments, such skincare products may be, and may have been, purchased by the user from another source. In some embodiments, the application may instruct the user to take photographs of her or his skin (preferably within a GUI tool allowing the user to take photographs with the user's smartphone provided within the mobile application) at particular times. The photographs taken are preferably labeled by time (e.g., in a calendar format), and a user may therefore review photographs of her or his appearance over the course of her or his skin management with the methods set forth in the present application. The user may also enter particular data into the application, including, but not limited to, water intake and other wellness-oriented products used by the user, to aid an administrator or the mobile application itself, in providing feedback to the user. In one embodiment, the control system issues personalized wellness recommendations (and, specifically, wellness recommendations relating to the user's skin care regimen) at regular intervals (e.g., daily, each morning) based at least in part on at least some data taken in from the user. In some embodiments, the exact nature and number of the cartridges sent to a user at regular intervals also may be altered, based in part on the data input by the user. In some embodiments, additional personal devices, aside from or in addition to the user's smartphone or PDA, may be used to provide data to the control system. For example, data may be imported by the control system from a personal activity monitor (such as a smartwatch or FITBIT brand activity monitor), to monitor the user's health and provide such recommendations. These and other aspects of the invention will be made clearer in reference to some of the particular exemplary steps set forth in FIG. 2, as discussed in further detail below.

Beginning with step 201, the control system first determines whether a new user has been enrolled into the system, meaning that she or he has provided identifying personal information and created a user account, in which personal data related to the user is stored, such that further actions may be taken by the control system in relation to the user and related to her or him (and her or his skin health). In some embodiments, the user may be required to provide data related to payment or other consideration for the services subsequently rendered, before the further steps provided herein are carried out. Assuming that a new user has been so enrolled, the control system may next proceed to step 203, in which additional personal data may be taken from the user (or, in some embodiments, a third party authorized by the user), related to the user. For example, the user may provide his or her age, address, and information related to her or his skin condition(s), through an intake procedure provided by the control system (e.g. a “windows” based GUI).

Next, the control system proceeds simultaneously to two separate tasks (for example, via a multi-threaded processor or multiple processors), as shown in steps 205 et seq., shown on the left-hand side of the figure, and steps 209 et seq., shown on the right-hand side of the figure.

In step 205, the control system may direct that an initial testing kit comprising swabs and a sampling and/or testing media be issued to the user. In some embodiments, such a kit comprises a set of cartridges comprising rollers, similar in nature to greater cartridge unit 115, set forth above. In other embodiments, such a kit may comprise another form of swab or testing media applicator, or a set thereof. Based on the initial personal data provided (e.g., a stated age and skin type), the control system may make the initial determination that a kit with a substrate and media suitable for testing and/or managing the user's skin be issued. Thus, in some embodiments, the control system may identify a particular set of media, matching that data, and suitable for managing the individual's skin (by age and skin type) in inventory, through a connected business/intranet subsystem, controlled by the control system. The control system may also direct that employee(s) or shipping machines arrange for the shipment of that initial set of testing media to the user, at her or his recorded home address, using commercial shipping providers. As the user begins to apply that shipped media to her or his skin (in some embodiments, exclusively to the skin of her or his face), she or he will then return the used cartridges, swabs or other samples, in step 207. This may be done in one shipment, for the entire supply provided in the initial shipment by the control system or, in some embodiments, a user may return the cartridges, swabs or samples on a rolling basis, one at a time, or in sets relevant to testing (such as, but not limited to, the plurality of test samples in a set as discussed above for some embodiments). To return the samples, the user may send the samples back, through the mail, to the address where the inventory controlled by the control system is kept (e.g., a warehouse under real-time inventory management by the control system).

In parallel, the control system may also be receiving the samples back from the user, and conducting testing to determine the user's skin condition(s) and health, and issuing regular (e.g., daily, every morning, via a mobile software application presented to the user on her or his smartphone or PDA) feedback and personal recommendations for enhancing or maintaining her or his skin health, in steps 209 and 211. In some embodiments, the control system manages employees and/or robotic or otherwise automated test equipment to process, test and analyze samples taken of the user microbiome and other factors related to her or his skin health and appearance. For example, in some more specific embodiments, the automated test equipment may include machines running polymerase chain reaction (“PCR”) analyses of the DNA and RNA genes of microbes taken from the user's microbiome. In more specific embodiments, quantitative polymerase chain reaction (“qPCR”) and/or 16S ribosomal RNA gene sequencing is carried out, creating a genetic profile of microbes within the user's microbiome. In still more specific embodiments, these techniques target the genomes of actinobacteria in the user's microbiome, from which the control system and/or a user may analyze aspects related to skin health. In some embodiments, qPCR is applied by the control system to assess the relative presence of a known gene using phylogenetic markers associated with actinobacteria, as a baseline offering selectable by the user for a lower, initial fee or other consideration. In some embodiments, qPCR is applied by the control system to assess the relative presence of a known gene using phylogenetic markers associated with bifidobacteria. In some embodiments, qPCR is applied by the control system to assess the relative presence of a known gene using phylogenetic markers associated with streptococcus. In some embodiments, qPCR is applied by the control system to assess the relative presence of a known gene using phylogenetic markers associated with any other known bacteria present in human microbiomes, and which may be suitable for the assessment of human microbiomes. Preferably, but not necessarily, in some of those embodiments, a user may pay an additional or higher fee and, once that additional or higher fee has been paid, the control system then also runs the 16S ribosomal RNA test on samples swabbed, reporting and using analyses related to that testing to produce additional swabs and testing and/or application media more particularly designated for managing and testing the user's skin, based on that analysis, as a premium service. More specifically, in some embodiments, the 16S ribosomal RNA test may be run from specific swabs, which the control system directs the user to apply to her or his face (or a part thereof, such as the forehead), excavating, and allowing the control system to investigate the total breadth of, bacteria that inhabit a customer's sebaceous and non-sebaceous skin regions. If such testing indicates a healthy, unhealthy or otherwise health- or appearance-relevant condition, the control system then may report those conditions to the user as feedback, in step 209, and give the user recommendations (e.g., reduce sodium or sugar intake, or increase hydration, exercise and cleansing) in step 211. In some embodiments, the control system requests that a user select a goal relevant to her or his skin health or appearance (e.g., through a GUI presented to the user). In some embodiments, after receiving and testing samples from such a user, the control system indicates to a user whether she or he is making progress toward that particular goal, based on results of that testing. In some such embodiments, if the user is making such progress, according to signs of skin health correlated with such a goal that are the subject of that testing, the control system reports that progress to the user. In some such embodiments, if the user is not making such progress, according to signs of skin health correlated with such a goal that are the subject of that testing, the control system reports that lack of progress to the user. For example, in some embodiments, the presence or count of bacteria of at least one particular species from one sample, taken from such a user's skin microbiome at one point in time, is compared to the presence or count of bacteria for that species in a second sample, taken from such a user's skin microbiome at a later point in time. Based on tests run on both samples, such as the types of DNA testing set forth in this application, and based on data correlating such presence and/or counts of bacteria to particular goals, the control system may then indicate whether the user is, or is not, making such progress toward such a goal. In some embodiments, the control system may issue additional recommendations to the user, based on such progress or lack of progress.

As another premium service, and in exchange for the user paying an additional fee, in some embodiments, any of the above testing methods may be applied to other parts of the user's body, to gather and analyze samples of the microbiome present on the skin of those parts of the user's body, in addition to areas of the user's face.

Turning to step 213, after steps 207 and or 211, the control system next may prompt the user (e.g., with a GUI screen on a connected smartphone created by a mobile application controlled by the control system, requesting or enabling data entry from a user) for data input related to the user's skin health. For example, in some embodiments, a user and/or the control system may manually enter data through an input device (e.g., keyboard or touchscreen of the smartphone) or allow for the passive entry of data (e.g., permitting the mobile application to access the smartphone's camera, to take photographs of the user's skin and/or overall appearance, or gathering data from other sensors connected to the smartphone or another PDA running a mobile application controlled by the control system—such as an activity monitor, such as a FITBIT.)

In subsequent step 215, the control system may next determine if any other entries or requests related to the software and GUI have been entered by the user. For example, if a user wishes, he or she may specifically request additional hydration, cleansing or other skin agents, even if the control system has not determined they are necessary, with a manual request through the GUI. If so, the control system may execute such requests, sending the requested tests, management techniques, or other actions, in step 217. In any event, whether or not such other entries or requests have been made, the control system eventually proceeds to steps 219 and 221, in which steps 207 through 217 are repeated, incorporating additional test results and other data created by those steps, to issue additional sets of cartridges based on those data—customized (“tuned”) to the user's skin profile, based on the personalized data and/or requests. Such subsequent repetitions of that series of steps may continue indefinitely (e.g., pending continued payments or account maintenance by the user) or may end at a particular time or number of rounds pre-selected by the user, an administrator and/or the control system. The control system then returns to the starting position.

As mentioned above, the above-referenced testing, recommendations, feedback and other steps may be conducted by the machines or human employees managed by the control system. In some embodiments, a human administrator or other agent may also, or alternatively, carry out some of the steps set forth in this application, in accordance with aspects of the present invention, as will be readily apparent to those of ordinary skill in the art to which this invention relates.

The embodiments set forth above are exemplary only of the wide range of options for managing cartridges and data related to testing a user's skin microbiome and managing his or her skin that will be readily apparent to a person of ordinary skill in the art implementing the present invention. No example provided in this application should be read as a limitation of any claim. The invention may be implemented in any suitable combination, and with any aspect set forth above, including other and/or more general forms and variations.

FIG. 3 is a schematic block diagram of some elements of an exemplary control system 300, preferably incorporating a machine-readable medium, that may be used to implement various aspects of the present invention, some of which aspects are depicted in the remaining figures of this application. The generic and other components and aspects described herein are not exhaustive of the many different systems and variations, including a number of possible hardware aspects and machine-readable media that might be used, in accordance with the invention. Rather, the system 300 is described here to make clear how aspects may be implemented.

Among other components, the system 300 includes an input/output device 301, a memory device 303, long-term, deep data storage media and/or other data storage device 305, and a processor or processors 307. The processor(s) 307 is (are) capable of receiving, interpreting, processing and manipulating signals and executing instructions for further processing and for output, pre-output and/or storage in and outside of the system. The processor(s) 307 may be general or multipurpose, single- or multi-threaded, and may have a single core or several processor cores, including microprocessors. Among other things, the processor is capable of processing signals and instructions for the input/output device 301, to cause a user interface to be provided or modified for use by a user on hardware, such as, but not limited to, a graphical user interface (“GUI”) provided by specialized software (e.g., a mobile application (“App”) on a smartphone or PDA, or PCR testing device, such as 319, and/or a personal computer monitor or terminal monitor with a mouse and keyboard and presentation and input-facilitating software (as in a desktop GUI) (311 or 319)).

For example, “window” presentation user interface aspects may present a user with the option to command other aspects of the control system to store and access data related to skin microbiome test results, from processing and analyzing a testing media provided on and scanned from a roller device, such as wand device 100, set forth above, and to issue personalized recommendations and new skin management techniques and sampling swabs and testing or application media to a user based on that data (and, in some embodiments, other user- and/or sensor-input data). The processor or processors 307 is/are capable of processing instructions stored in long-term, deep data storage media and/or other data storage device 305 and/or memory device 303 (or ROM or RAM), and may communicate via system buses 375. Input/output device 301 is capable of input/output operations for the system, and may include and communicate through innumerable input and/or output hardware, and innumerable instances thereof, such as a computer mouse, test media processing device or other sensors, actuator(s), communications antenna, keyboard(s), smartphone(s) and/or PDA(s), networked or connected additional computer(s), camera or microphone, a mixing board, reel-to-reel tape recorder, external hard disk recorder, additional movie and/or sound editing system or gear, speakers, external filter, amp, preamp, equalizer, computer display screen or touch screen. Such a display device or unit and other input/output devices could implement a program or user interface created by machine-readable means, such as software, permitting the system and user to carry out the user settings and input discussed in this application. 301, 303, 305, and 307 are connected and able to send and receive communications, transmissions and instructions via system busses 375. Long-term, deep data storage media and/or other data storage device 305 is capable of providing mass storage for the system, and may be a computer-readable medium, may be a connected mass storage device (e.g., flash drive or other drive connected to a U.S.B. port or Wi-Fi) may use back-end (with or without middleware) or cloud storage over a network (e.g., the Internet) as either a memory backup for an internal mass storage device or as a primary memory storage means, or may simply be an internal mass storage device, such as a computer hard drive or optical drive. Generally speaking, the system may be implemented as a client/server arrangement, where features of the invention are performed on a remote server, networked to the client and made a client and server by software on both the client computer and server computer. Also generally speaking, the system may provide output and other services to an external system, including any of devices and auxiliary devices and/or systems 309-319. Similarly, system 300 is capable of accepting input from any of those devices and auxiliary devices and/or systems 309-319, and modifying stored data within them and within itself, based on any input or output sent through input/output device 301.

Input and output devices may deliver their input and receive output by any known means, including, but not limited to, any of the hardware and/or software examples shown as 309-319.

While the illustrated system example 300 may be helpful to understand the implementation of aspects of the invention, any suitable form of computer system known in the art may be used—for example, a simpler computer system containing just a processor for executing instructions from a memory or transmission source. The aspects or features set forth may be implemented with, and in any combination of, digital electronic circuitry, hardware, software, firmware or any other computing technology known in the art, any of which may be aided with external data from external hardware and software, optionally, by networked connection, such as by LAN, WAN or the many connections forming the Internet. The system can be embodied in a tangibly-stored computer program, as by a machine-readable medium and propagated signal, for execution by a programmable processor.

The method steps of the embodiments of the present invention may be performed by such a programmable processor, executing a program of instructions, operating on input and output, and generating output and stored data. A computer program includes instructions for a computer to carry out a particular activity to bring about a particular result, and may be written in any programming language, including compiled and uncompiled and interpreted languages and machine language, and can be deployed in any form, including a complete program, module, component, subroutine, or other suitable routine for a computer program.

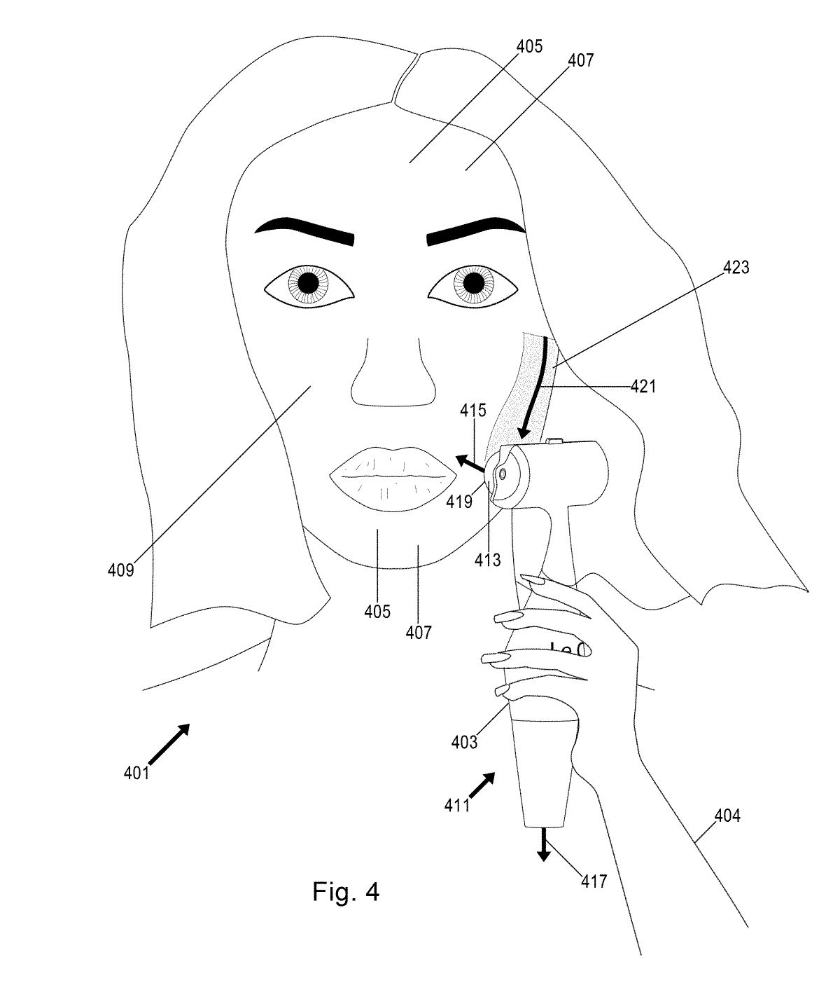

FIG. 4 is a perspective drawing depicting a user 401 and specialized hardware carrying out aspects of a method of sampling and managing human skin and a microbiome, according to some embodiments of the present invention. More specifically, user 401 is performing a swabbing of her facial skin and its microbiome, to enable the testing of DNA, as discussed in greater detail below. As with any other example embodiment of the invention, the example embodiment pictured in the present figure is for convenience, to aid the reader in understanding aspects of the invention. Numerous other embodiments of the invention are also possible, as will be readily apparent to those of ordinary skill in the art. For example, in some embodiments, a more conventional sampling swab for sampling DNA from a human skin may be used to sample a human microbiome.

The user 401 is shown gripping that hardware—namely, an example wand device 403—in her left hand 404, and sampling the microbiome 405 of the epidermis 407 of her face 409. Wand device 403 may be, or may be similar to, wand device 100, depicted in FIG. 1, above. By gripping handle grip section 411 (which, in some embodiments is the handle grip section 102 of FIG. 1, above) and pressing sampling roller 413 (which, in some embodiments is sampling and/or application roller 107 of FIG. 1, above) against her epidermis 407 of her skin, as shown by force or motion vector arrow 415, user 401 may begin to take a sample of microbiome 405. In some embodiments, by pulling downward on handle grip section 411, in the direction shown by force or motion vector arrow 417, while maintaining pressure from sampling roller 413 against her epidermis 407, sampling roller 413 begins to rotate, clockwise, in the perspective of the figure. As sampling roller 413 rotates, its outer surface 419 remains in contact with the epidermis 407 of her face 409 (e.g., due to the force of stiction), and generally does not drag across her epidermis 407. Instead, outer surface 419 of sampling roller 413 maintains a laterally stationary, a.k.a., a 1-to-1 (one-to-one), surface-to-surface contact ratio with epidermis 407 in a direction along the outer surface of epidermis 407, with which sampling roller 413 is in contact. As a result, the outer surface 419 of sampling roller 413 follows a straight, or approximately straight path 421 along the outer surface of user's epidermis 407 of her face 409, in a generally downward direction along user's face 409, covering a sampling or application area, such as the example shown as 423, of epidermis 407.

In some embodiments, user 401 may not, and may be instructed not to, break contact between epidermis 407 and sampling roller 413 as she or he is performing a swabbing of her epidermis 407 with wand device 403 to cover a sampling or application area, such as the example shown as 423. In some such embodiments, user 401 may not, and may be instructed not to, over-rotate sampling roller 413 in other words, not to sample different areas of epidermis 407 with sampling roller 413 than that instructed, or go over the same area more times when sampling. In this way, over-sampling or redundant sampling of bacteria or other cultures or matter may be avoided. In other embodiments, a user may be given a time limit in which to perform continuous swabbing of a sampling or application area of her epidermis 407, such as example application sampling or application area 423.

In some embodiments, any form of swab known in the art for sampling aspects of a microbiome, other than sampling roller 413 and wand device 403 generally, may instead be used to perform the swabbing of a sampling or application area of her epidermis 407. For example, in some embodiments, a linear swab with a rod-shaped handle and a wad of substrate on one end may be used to sample the microbiome. As with sampling rollers set forth in this application, such a wad of substrate may be supplied with testing or application media, loaded onto it, in some embodiments. However, also as with sampling rollers set forth in this application, such a linear swab may be supplied dry, in some embodiments (for example, without such media, or, as another example, with such media supplied in a separate container). Some such embodiments are discussed in greater detail, below.

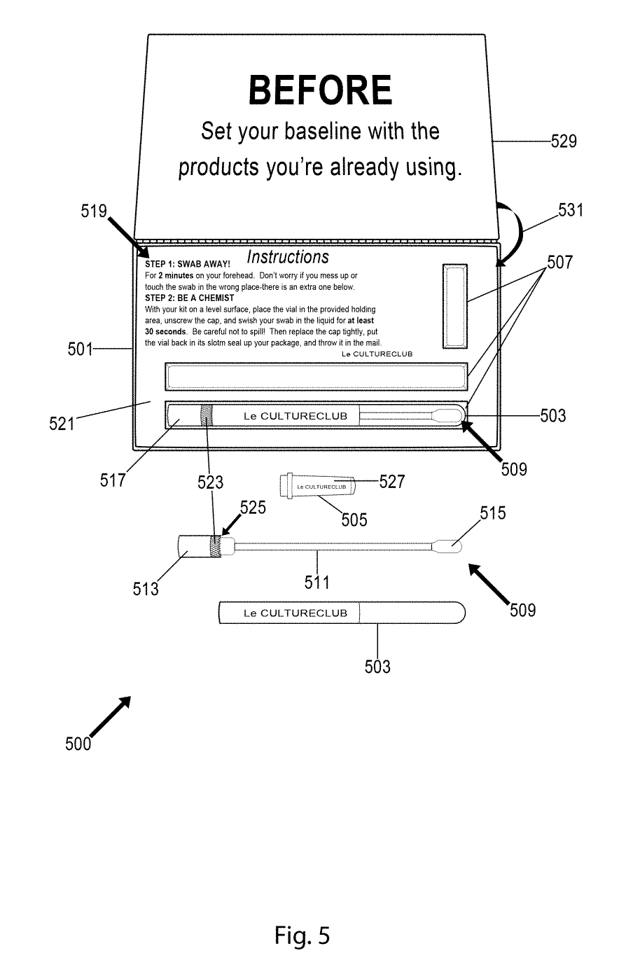

FIG. 5 depicts an example kit 500 which may be issued to, and used by, a user of a skin testing and management system and method, in some embodiments of the present invention.

As explained in greater detail above, in reference to FIGS. 2 and 3, in some embodiments of the invention, an administrator of a skin testing and/or management system issues a kit, such as example kit 500, to a user after that user has joined a program for skin testing and management managed by the administrator. In some embodiments, the administrator may be, or may include, a computer hardware- and software-based control system, such as the example control system provided in reference to above, running specialized software for the management of a program for skin testing and management.

Kit 500 may include a shipping box 501, variably enclosing various hardware for sampling, testing and/or managing a user's skin. In some embodiments, kit 500 may include one or more swabbing, testing or application hardware sub-containers, such as example swab packaging tubes 503, and example reagent-containing vial 505. In some embodiments, such a vial may not be included. In some such embodiments, reagents or other testing or application media may be provided within swabs or other testing hardware. In some embodiments, fewer or more swab packaging tubes may be included within shipping box 501. In some embodiments, shipping box 501 may include internal cutouts or padding 507, to protect such swabbing, testing or application hardware during shipment. Swab packaging tubes 503 may contain a skin sampling swab, such as example sampling swabs 509, in some embodiments. In some such embodiments, an example swab, such as those shown as 509, includes a rigid rod 511, connected with a handle 513, at one end, and a wad 515 of sampling substrate, at the other end. As mentioned above, the sampling substrate of sampling swabs 509 is preferably a flocked nylon substrate, suitable for taking samples of a human skin microbiome. In some such embodiments, handle 513 is also a tube end cap, such as the example shown as 517. Tube end caps 517 may serve to enclose a sampling swab, such as sampling swab 509, once it is placed within a swab packaging tube, such as the examples shown as swab packaging tubes 503.

In some embodiments, a user of the kit 501, and the program for skin testing and management, may be provided with instructions for carrying out swabbing, testing and/or application. In some embodiments, a user of such a program may be provided with instructions for carrying out swabbing using a swab, such as example sampling swabs 509, on the user's face. In some such embodiments, such instructions are provided within kit 500, for the user to view, hear or read. For example, in some embodiments, the user is provided with written instructions 519, located on an interior surface 521 of shipping box 501.

Those instructions provided to the user, such as written instructions 519, may instruct a user to open kit 501 and one of swab packaging tubes 503, by gripping one of handles 513, and pulling it away from one of swab packaging tube 503, breaking one of swab seals 523. In some embodiments, swab seals 523 irreversibly reveal whether they have been broken by such opening of swab packaging tubes 503, or other tampering, for example, by exhibiting a torn pattern 525. The instructions may then direct the user to swab his or her face by touching a wad, such as 515, of a sampling swab, such as either of sampling swabs 509, to an area of his or her face. In some embodiments, such an area of the user's face may be defined in the instructions. For example, in some embodiments, the user may be instructed to swab skin on his or her forehead, and on his or her forehead only. In some embodiments, the length of time during which such swabbing shall take place is defined in the instructions. For example, in some embodiments, a user may be instructed to swab the subject area (e.g., skin on his or her forehead) for two consecutive minutes. In other embodiments, any other time period suitable for obtaining a critical mass of microbiome constituents as a sample for testing may be used.

The instructions also may direct the user, once such swabbing has been completed, to douse wad 515 with a testing reagent or other material 527, within reagent-containing vial 505. In some embodiments, testing reagent or other material 527 may include a lysis buffer. In some embodiments, testing reagent or other material 527 may include a lysis solution. In some embodiments, no such reagent-containing vial 505, or testing reagent or other material 527, is included within kit 500, and a user may simply swab his or her microbiome with one or more of sampling swabs 509, and return them, as discussed in greater detail, below. In some embodiments, multiple reagents or other materials may be provided to a user for dousing a sampling swab, such as the sampling swabs shown as sampling swabs 509. The instructions may provide that the user to douse or immerse wad 515 for a particular length of time, among other things. In some embodiments, the user may be directed to repeat the swabbing and testing steps set forth above for one of swabs 509, with another of sampling swabs 509. For example, in some embodiments, the user may be directed to so repeat swabbing and testing with another of sampling swabs 509 if the user has contaminated the wad of one of sampling swabs 509 for example, if the user has inadvertently dropped one of sampling swabs 509 on the floor.

In some embodiments, the instructions may direct the user to then return any of wad, such as wad 515, once successfully doused, to one or more of swab packaging tubes 503. However, in some embodiments, as mentioned above, the user need not douse wad 515 prior to returning any of sampling swabs 509. In some embodiments, the instructions may direct the user to place any of sampling swabs 509 which now contain samples from swabbing and have been successfully doused (and now contain analytes) along with the tube(s) in which they have been returned, along with testing reagent or other material 527, as described above into shipping box 501 of kit 500, close shipping box 501 (e.g., by swiveling a hinged lid 529 onto a top of shipping box 501, as shown by swiveling motion arrow 531) and to ship it back to the administrator, for further testing and analysis of the analytes.

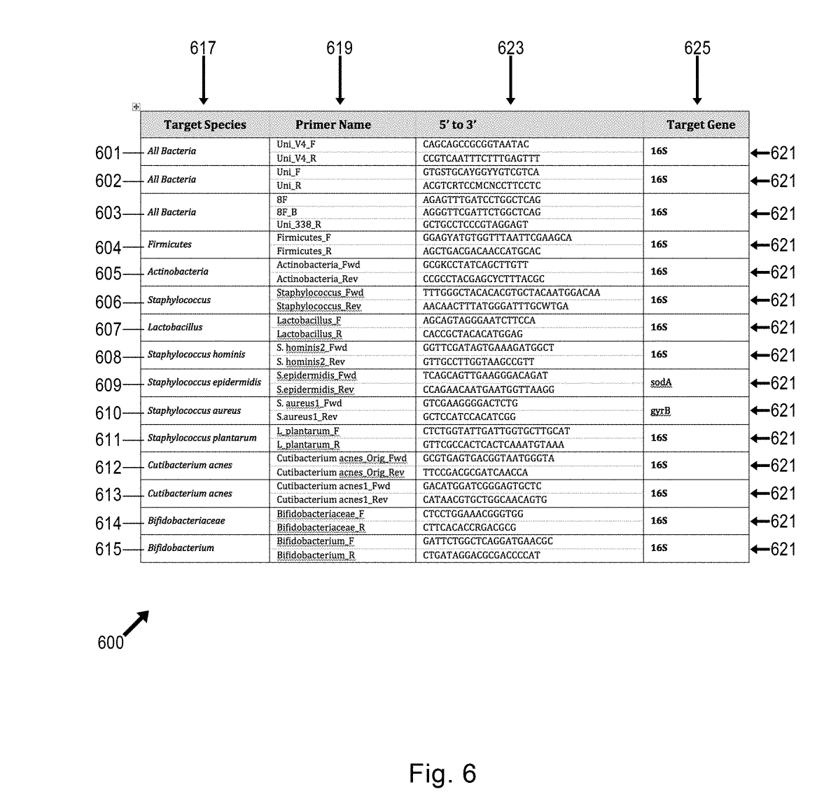

FIG. 6 depicts a table 600, including coding and other aspects of an example array of primers for DNA testing of a sample taken from a human skin microbiome, according to some embodiments of the present invention.

In some embodiments, swabs, such as sampling swabs 509, discussed above, containing samples from a human skin microbiome, such as example microbiome 405, discussed above, are received and processed by an administrator of a skin testing and/or management system. For example, in some embodiments, labels on such swabs (or protective containers containing such swabs) identify the user and sample taken, and the administrator may run analytical testing on those samples, to determine the makeup of the user's skin microbiome. For example, in some embodiments, DNA testing is performed, and the DNA or RNA of particular microorganisms are the analytes within those samples. In some embodiments, the DNA of particular bacteria within human microbiomes are the analytes tested by the administrator. As mentioned above, in some embodiments, that administrator may be, or may comprise, a control system comprising computer hardware and software, such as the example control system set forth above, in reference to FIG. 3.

In some embodiments, specific species of bacteria are the analytes tested by the administrator in testing and analysis. For example, in some embodiments, the administrator tests for the presence of any of the species of bacteria, or group of species of bacteria, identified in data cells 601 through 615, in column 617 of table 600. In other embodiments, the administrator may test for the number of any of those species of bacteria. In still other embodiments, the administrator may test for the proportion of any of those species of bacteria, in comparison to any other species of microorganism(s) present in the sample. In still other embodiments, the administrator may compare the number or proportion or any other suitable datum based on the sample to any suitable comparator.

According to some embodiments, the presence or proportion of the bacterial species tested, such as any of the species of bacteria identified in data cells 601 through 615, may be tested by combining genetic primers with at least part of the sample, allowing the primers to attach to genetic material from microorganisms within the sample. In some embodiments, those primers may be any or all of the primers identified with the names shown in data cells within column 619 of table 600. Any of the primer name(s) shown in the same row, such as any of rows 621 of table 600, as a particular species of bacteria, is a suitable primer for attaching to, and testing for the presence and/or number of, that particular species of bacteria. Similarly, the nucleotide sequences shown in column 623 are the genetic sequences of the primers provided in column 617, and any of the sequences shown in the same row, such as any of rows 621 of table 600, as a particular primer is a correct sequence for that particular primer.

Finally, in column 625, the target genes associated with the primers named in column 619 are provided. Similarly, any of the gene name(s) shown in the same row, such as any of rows 621 of table 600, as a particular species of bacteria, primer, and genetic sequence is the name of the gene targeted by those primers and genetic sequences.

As mentioned above, in some embodiments, PCR testing is conducted on the samples to amplify and test for the presence and relative prevalence of the DNA of particular species of bacteria, such as any of the species of bacteria set forth in table 600. In some embodiments, that PCR testing is conducted using the primers set forth in column 619 and the associated genetic sequences set forth in column 623. In some embodiments, qPCR testing is conducted, and a fluorescent binding dye, such as SYBR GREEN may be used to monitor the emerging yield curve of the qPCR reaction. In other embodiments, any suitable markers, dyes or other reagents used PCR or qPCR may be used. Such qPCR testing may be carried out in the following stages: 1. a “Hold Stage,” in which the analyte DNA from the sample is denatured at 50 degrees Celsius for two (2) minutes, followed by an increase in temperature to 95 degrees Celsius for ten (10) minutes; 2. a “PCR Stage,” in which DNA amplification takes place for several cycles (e.g., forty (40) cycles), at ninety-five (95) degrees Celsius for fifteen (15) seconds, followed by a decrease in temperature to sixty (60) degrees Celsius for one (1) minute; and 3. a “Melt Curve Stage,” in which results are quantitatively, during reaction, and derived from a melt temperature, or the temperature at which one-half (½) of the DNA being tested has denatured, at a temperature of (95) degrees Celsius for fifteen (15) seconds, followed by a decrease in temperature to sixty (60) degrees Celsius for one (1) minute, followed by an increase in temperature to (95) degrees Celsius for fifteen (15) seconds. Any or all PCR or qPCR steps may be carried out on a computer-hardware and software-based control system, and run on a chip substrate within DNA analytical hardware of that control system (which may be a control system such as the control system set forth above in reference to FIG. 3.)

The example species of bacteria, primers, genetic sequences and target genes set forth in FIG. 6 are a single example array for genetic testing of a microbiome, in accordance with some embodiments of the present invention. This one example is for ease of understanding the present invention, and is not exhaustive or limiting. Many other arrays, with more, or less, species of microorganisms tested, and different primers, genetic sequences and target genes tested, may also be used, in various embodiments of the present invention. In some embodiments, any suitable array of species of microorganisms, primers, genetic sequences and target genes found on human skin may be used to test and analyze a microbiome.

As a result of the testing procedures set forth above, the administrator may create a skin microbiome profile, based on the presence or number of particular bacterial species isolated the sample taken from the user's epidermis, as discussed above. Based on the test results, or that profile, the administrator may match off-the-shelf skin-care products, which are determined to be most suitable for the user, based on those results, or that profile. The administrator may then send such matched products to the user, for application to his or her epidermis, managing his or her skin microbiome and skin, in some embodiments. In other embodiments, the administrator may provide computer-based links or other information enabling the user to purchase matched products her or himself (e.g., through another retailer). In some embodiments, the user may disclose to the administrator the particular skin care products that he or she is currently using, and the control system may compare the results of the testing set forth above with data concerning those skin care products. In such embodiments, the control system and/or administrator may recommend changes to the user's skin care regime, such as the elimination of particular skin care products used by the user, based on those comparisons, as well as issue skin care products to the user. Those recommendations may be based, at least in part, on a list of skin care products and data associated with the skin care products, indicating whether those skin care products may be problematic or helpful for a particular user with particular test results, or ranges of test results. In some embodiments, those recommendations are based on wellness goals of the user. In some such embodiments, those recommendations are based on skin care goals of a user. For example, a user may have a skin care goal, and the administrator may issue products and recommendations to serve that goal, of eliminating an adverse skin condition. Examples of such skin conditions that may be the subject of such goals may include, but are not limited to, acne, rosacea, oil overproduction, and insufficient hydration. In some embodiments, the administrator will send the recommendations, in a written form, along with skin care products, to the user.

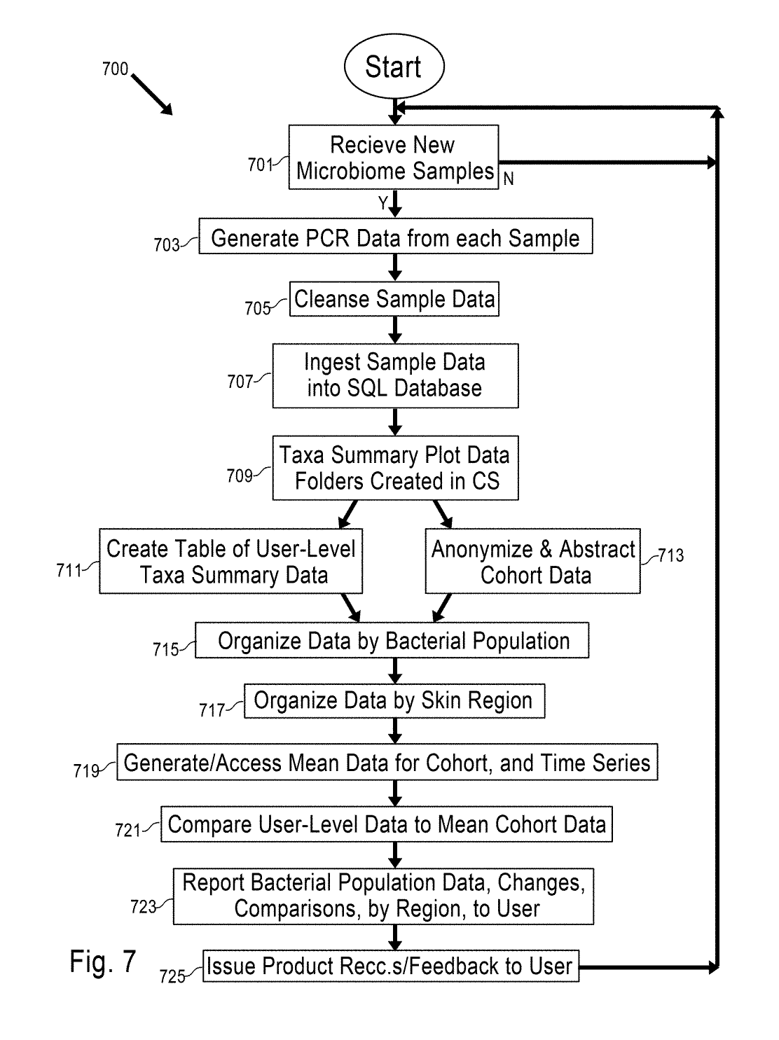

FIG. 7 is a process flow diagram, setting forth several example steps 700 that may be undertaken by a user and/or control system (such as the control system set forth above, in reference to FIG. 3) implementing some skin microbiome analysis and skin management embodiments of the present invention.

As mentioned above, in some aspects of the invention, a control system monitors results of aspects of the invention and delivers feedback to a user, in various embodiments. In some embodiments, as set forth above, such a control system enrolls a user of the skin microbiome analysis and skin management system periodically sends a set of swabs (e.g., in a testing kit) to a user, and instructs that user to swab her or his skin microbiome, producing at least one sample. Assuming steps such as those have taken place for a number of users, which may, in some embodiments, be organized into a group of user's (referred to herein as a “cohort”) the control system may take additional steps, as set forth below.

Beginning with step 701, the control system first determines whether it has received new samples of a user's microbiome, for example, by the introduction of the sample into DNA analytical hardware within the control system. If so, the control system proceeds to step 703, in which it runs at least one PCR test, generating data related to the results of the PCR test. For example, in some embodiments, such a sample is processed by a PCR chip substrate within DNA analytical hardware of the control system, yielding statistical data regarding the presence and prevalence of various species of bacteria, or other microorganisms, present on the user's skin microbiome (e.g., in a region thereof, such as the forehead, chin, etc.) In some embodiments, those data related to the results of the PCR test are then cleansed, in step 705, prior to being ingested into a database within, or accessible to, the control system, in step 707. In some embodiments, such a database may be an SQL database. In other embodiments, such a database may be in any suitable form of database for organizing PCR-related data. Upon that data ingestion, the control system may create folders, or otherwise store, data on the presence and prevalence of each of several bacteria within the sample submitted by the user (“taxa summary plot data”), in step 709. For example, in some embodiments, taxa summary plot data may include a count, density or other measure of the number or prevalence of individuals of each bacterial species tested for by the control system. In some embodiments, such bacterial species tested for may include any or all of the following bacteria: Firmicutes, Actinobacteria, Staphylococcus, Lactobacillus, Staphylococcus hominis, Staphylococcus epidermidis, Staphyococcus aureus, Lactobacillus plantarum, Cutibacterium acnes, Bifidobacteriaceae, and Bifidobacterium. However, in some embodiments, the presence of other bacteria may also, or in addition, be tested for.

The control system may create a table of the taxa summary plot data for such a user, in step 711, showing the presence, count and/or density of each species of bacteria found in the user's sample in the PCR test. In parallel, the control system may anonymize and abstract that same data, in step 713—for example, by using a unique code, with no personal identifying information regarding the user to whom the data relates—and organize it with the data of other users, e.g., in another data set related to a cohort of users.

All of these data related to the user, and/or to a cohort of users, may then be further processed, as follows, by the control system. In step 715, the control system may organize data related to each bacterial population tested for. For example, with respect to the user's taxa summary data, the control system may list each bacterial species tested for, and the presence, count and/or density of individuals thereof found in the user's sample. As another example, with respect to all of the anonymized, abstracted data from an entire cohort of users, the control system similarly may list each bacterial species tested for, and the presence, count and/or density of individual bacteria thereof found in each cohort user's sample(s). Similarly, in step 717, the control system may organize data related to each skin region swabbed and tested. For example, with respect to the user's taxa summary data, the control system may list each skin region tested, and the presence, count and/or density of bacteria found in the user's sample taken from that skin region. As another example, with respect to all of the anonymized, abstracted data from an entire cohort of users, the control system similarly may list each skin region tested, and the presence, count and/or density per sample related to each region found in each cohort user's sample(s). The control system may then proceed to further analyze these data, and/or lists thereof, in step 719. For example, in some embodiments, the control system generates average data (e.g., mean data) based on the listed anonymized, abstracted data from the entire cohort of users. As a more specific example, in some embodiments, the control system may generate average (e.g., mean) counts and/or densities of bacteria of each bacterial species, and average (e.g., mean) counts and/or densities of bacteria found in each region of users' skin, in such analysis. These average data from the cohort may then be compared to the counts and/or densities of bacteria of each bacterial species and counts and/or densities of bacteria found in each region of the user, in step 721.

The results of these analyses and/or comparisons may then be reported to the user, in step 723. For example, if the user's sample indicates a higher or lower count or density of particular species of bacteria than the average for the cohort, that fact may be reported to the user. As another example, if the user's sample in sample indicates a higher or lower count or density of bacteria in the region tested by the sample, that fact may be reported to the user. In some embodiments, if the user has submitted a previous sample, previously tested and by the control system in the same way as the present sample, the results of those previous tests may be similarly compared to the results of the present test, and the control system may issue reports regarding the difference (i.e., change) in count and/or density of particular species of bacteria, or count and/or density of bacteria of the tested region, based on the user's taxa summary data.

Finally, and as discussed elsewhere in this application, the control system may issue recommendations to the user based on the data, analyses and comparisons set forth above. For example, in some embodiments, the control system may recommend a particular skincare product to the user. As another example, in other embodiments, the control system may recommend the discontinuation of a skincare product for the user. In some embodiments, the control system may recommend lifestyle changes to the user. In some embodiments, the control system may facilitate the purchase of such products by the user. For example, in some embodiments, the control system may provide a link (e.g., within a GUI of a web-based software application) to a retailer's listing for the recommended product. In other embodiments, the control system may present a GUI enabling the user to purchase the recommended product from the control system.

What is claimed is:

1. A system for sampling a skin microbiome, comprising:
a wand device, comprising a handle section and a rolling applicator section, wherein said rolling applicator section comprises a cartridge holder configured to receive and hold a cartridge unit comprising a roller comprising a rounded surface;
said cartridge unit and said roller, wherein said roller is rotatably fastened to said cartridge unit, and wherein said roller comprises a testing and/or application media on said rounded surface.

2. The system for sampling a skin microbiome of claim 1, wherein said testing and/or application media is capable of sampling bacteria of a microbiome of human skin by contact pressure.

3. The system according to claim 1, wherein the system comprises:
a control system, comprising computer hardware and software, wherein said control system comprises D.N.A. testing hardware, and wherein said D.N.A. testing hardware is configured to receive at least one sample from said testing and/or application media, and to test for the presence of particular species of bacteria.

4. The system according to claim 1, wherein said cartridge unit comprises at least one convex surface, and wherein said cartridge holder comprises at least one concave surface, and wherein said concave surface is complementary to said convex surface.

5. The system according to claim 1, wherein said cartridge unit comprises a projecting tab.

6. The system according to claim 1, wherein said sampling wand is marked with information identifying a user of said wand.

7. The system according to claim 3, wherein said wand device is marked with information identifying a user of said wand.

8. A method for managing a skin microbiome, comprising the following steps:
issuing a kit to a user of a skin management system, wherein said kit comprises a wand device, comprising a handle section and a rolling applicator section, wherein said rolling applicator section comprises a cartridge holder configured to receive and hold a cartridge unit comprising a roller comprising a rounded surface, and wherein said kit comprises said cartridge unit and said roller, wherein said roller is rotatably fastened to said cartridge unit, and wherein said roller comprises a testing and/or application media on said rounded surface.

9. The method for managing a skin microbiome of claim 8, wherein said testing and/or application media is capable of sampling bacteria of a microbiome of human skin by contact pressure.

10. The method for managing a skin microbiome of claim 8, comprising the following additional step:
receiving a sample of a human skin microbiome from said user, wherein said sample is obtained with said wand device.

11. The method for managing a skin microbiome of claim 9, wherein said rounded surface comprises a substrate.

12. The method for managing a skin microbiome of claim 11, wherein said rounded surface comprises a substrate comprises a flocked material.

13. The method for managing a skin microbiome of claim 12, wherein said flocked material comprises nylon.

14. The method for managing a skin microbiome of claim 10, comprising the following additional step:
running a genetic test on said sample.

15. The method for managing a skin microbiome of claim 14, wherein said genetic test comprises a polymerase chain reaction.

16. The method for managing a skin microbiome of claim 14, wherein said genetic test comprises quantitative polymerase chain reaction.

17. The method for managing a skin microbiome of claim 14, wherein said genetic test includes an analysis of a count or density of individuals of at least one species of microorganism within said sample.

18. The method for managing a skin microbiome of claim 14, wherein said at least one species of microorganism comprises at least one species of bacteria.

19. The method for managing a skin microbiome of claim 18, comprising the following additional step:
issuing a skincare product to said user, based on results of said genetic test.
